(19) United States
(12) Patent Application Publication
(10) Pub. No.: US 2003/0021835 A1
(43) Pub. Date: Jan. 30, 2003

(54) Medical patch with burstable partition

(75) Inventor: Anthony C. Pagedas, Greendale, WI (US)

Correspondence Address: Ryan Kromholz & Manion, S.C., P.O. Box 26618, Milwaukee, WI, 53226, US

(73) Assignee: Ancel Surgical R&D, Inc.

(21) Appl. No.: 09/911,770

(22) Filed: Jul. 24, 2001

(51) Int. Cl. A61K 9/70

(52) U.S. Cl. 424/449

(57) ABSTRACT

A dermal or transdermal dosage unit for administering a dosage of a pharmaceutical to the skin of a patient. The dosage unit includes a backing layer, which is non-permeable with respect to the pharmaceutical, a biologically acceptable adhesive, an impermeable coating, a compartment, and an impervious burstable membranous partition dividing the compartment into at least two subcompartments. Each subcompartment may contain a different medical compound. Upon rupture, the fragmented partition includes distal end portions which serve to aid in agitation of the mixture of compounds thereby forming the dosage pharmaceutical.

### BACKGROUND OF THE INVENTION

Transdermal absorption units have been developed for use with a variety of pharmaceutical products, including beta-blockers, estrogen replacements, and nitroglycerin. An example of a dosage unit has been disclosed in U.S. Pat. No. 6,221,384 granted to the applicant of the present invention, and U.S. Pat. No. 4,666,441 granted to Andriola et al. As disclosed in the Andriola reference, a dermal or transdermal patch includes a plurality of reservoirs, each reservoir containing at least one drug or drug formulation. The reservoirs disclosed in the Andriola reference are discrete compartments, which may or may not permit interaction of drugs in adjacent reservoirs. In those embodiments permitting adjacent drugs to be mixed, there exists difficulty for the user in ascertaining whether the respective compounds are adequately mixed prior to application.

### SUMMARY OF THE INVENTION

In view of the above-noted concerns, and also to present a dosage unit capable of storing multiple pharmaceutical components prior to mixing them at the time of use, the present invention teaches a novel dosage unit for transdermal and dermal absorption dosage units.

A conventional dosage unit comprises a removable backing layer that is impervious to the pharmaceutical product(s) to be administered, an adjoining layer having dissolved or microdispersed pharmaceutical product therein, a biologically suitable adhesive means by which the dosage unit adheres to the skin of the patient receiving the dosage, and an impermeable coating. The present invention further contemplates at least one continuous, uninterrupted, burstable membrane for adjacently retaining pharmaceutical components having differing compositions.

The pharmaceutical components are separated from one another along a predetermined portion by a continuous, uninterrupted, burstable membrane such that, at time of use, the membrane may be manually burst to allow the differing pharmaceutical components to intermingle and mix. This feature is useful in applications that require pharmaceutical ingredients to be mixed immediately prior to use, such as compounds that become activated only upon combination. The burstable membrane is preferably substantially impervious to the pharmaceutical components such that contact of the components does not occur until the membrane is manually burst. Further, after having been burst, the fragmented membrane facilitates complete mixture of the components by providing distal end portions that serve as a flagellating means while the dosage unit is manually massaged. This feature represents a departure from known, compartmented systems that allow little or no agitation of the compounds.

Additionally, the pharmaceutical components to be mixed may be provided with pigmentation to provide a visual indication of adequate mix. For example, a first component may be yellow and a second component blue, so that upon mixing, the dosage pharmaceutical appears as green. This visual reference allows the user to identify appropriate mix time.

The invention further contemplates a method of administering a controlled amount of a pharmaceutical to the skin of a patient by dermal or transdermal adsorption when the pharmaceutical to be administered is comprised of at least two pharmaceutical compounds that must be premixed immediately prior to administration. The method includes the steps of providing an absorption dosage unit having a removable backing layer, which is substantially impervious to the pharmaceutical to be administered. The dosage unit further includes a biologically acceptable adhesive layer for securing the dosage unit to the patient, a membranous layer permeable to the pharmaceutical and containing a measured amount of the pharmaceutical, a protective coating layer which is substantially impervious to the pharmaceutical, and a continuous, uninterrupted, burstable dividing means for dividing the pharmaceutical-containing layer into compartments. The burstable means is preferably substantially impervious to the pharmaceutical components to be mixed. Further steps include manually bursting the dividing means and manipulating the dosage unit such that the previously separated pharmaceutical components are mixed and intermingled to form the dosage pharmaceutical, removing the backing layer from the dosage unit to expose the adhesive layer and membranous layer, adhesively attaching the exposed layer to the skin of a patient, and allowing the membranous layer to remain adhesively attached to the skin of a patient for a predetermined amount of time.

### DESCRIPTION OF THE DRAWINGS

FIG. 1 is a top plan view of the dosage unit of the present invention and showing the burstable partition in phantom.

FIG. 2 is a cross sectional view of the dosage unit shown in FIG. 1 and taken along line 2-2 thereof.

FIG. 3 is a perspective view of the dosage unit showing the burstable partition while being burst and showing the pharmaceutical components in contrasting shading prior to being mixed.

FIG. 4 is a perspective view of the dosage unit showing manual manipulation thereof and intermingling of the pharmaceutical components.

FIG. 5 is a perspective view of the dosage unit affixed to a patient's arm.

### DETAILED DESCRIPTION

Although the disclosure hereof is detailed and exact to enable those skilled in the art to practice the invention, the physical embodiments herein disclosed merely exemplify the invention which may be embodied in other specific structure. While the preferred embodiment has been described, the details may be changed without departing from the invention, which is defined by the claims.

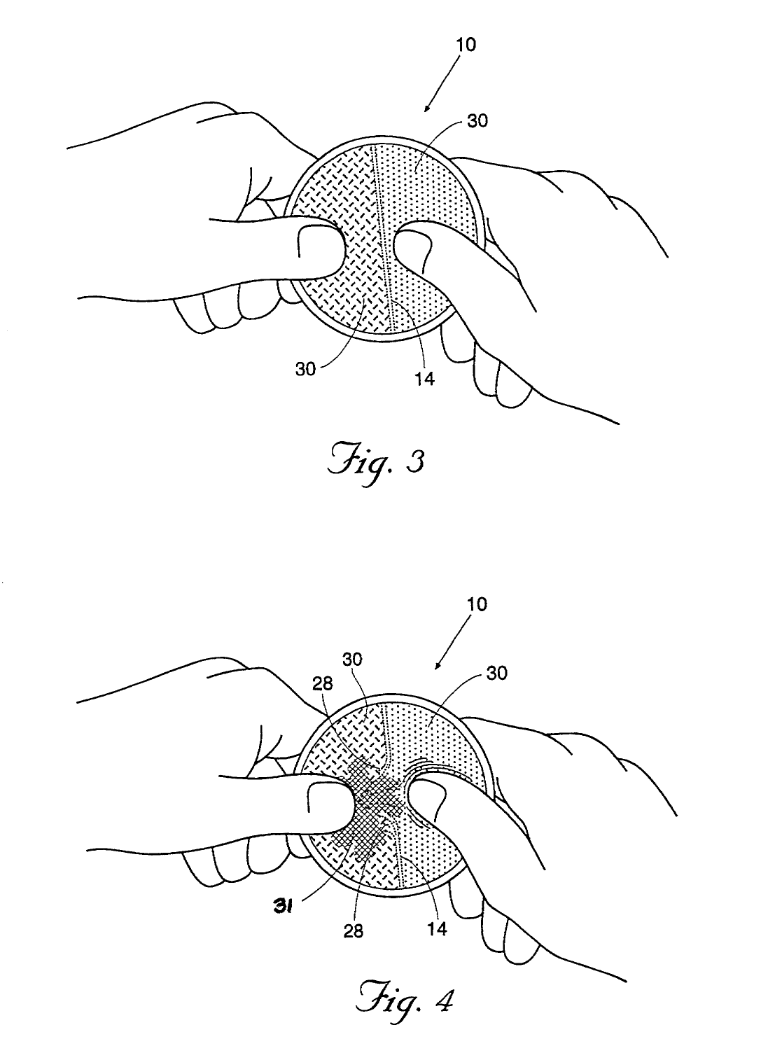

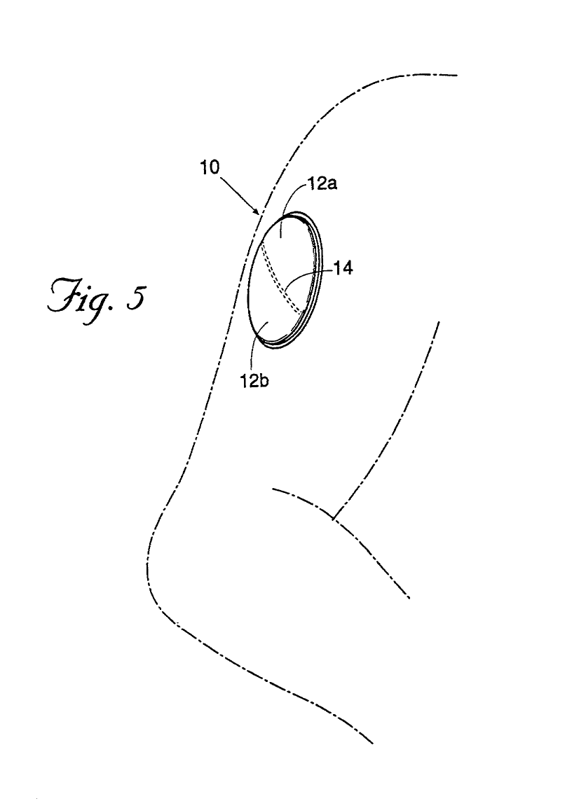

The present invention is directed to an improved dermal or transdermal dosage unit having a plurality of pharmaceutical components dispersed on a permeable membrane and adapted to be applied directly to the skin of a patient (see FIG. 5) receiving a mixed dosage pharmaceutical. The improvement resides principally in the continuous, uninterrupted burstable membrane 14 separating the pharmaceutical components 30 from one another prior to use, and in the feature of the burst membrane including flagellating means to aid in mixing of the various pharmaceutical components 30. Further, as seen in FIGS. 3-4, the pharmaceutical components 30 are preferably distinctly pigmented so that as the components 30 are mixed, a secondary color is created to indicate complete mixing.

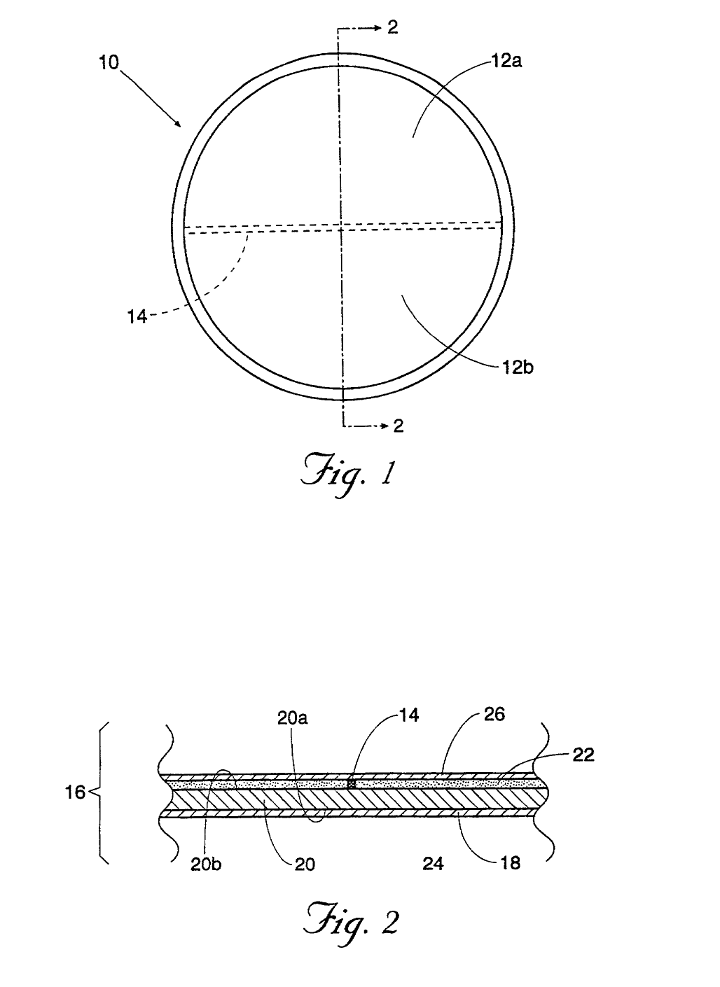

With reference to FIG. 1, the present invention, seen as a dermal or transdermal dosage unit, is generally indicated by reference numeral 10. The dosage unit or patch 10 includes a pharmaceutical dosage layer which is preferably divided into multiple sub areas 12a, 12b by way of a continuous, uninterrupted, burstable, partition or membrane 14. The membrane 14 is preferably substantially impervious to the pharmaceutical components 30. The dosage unit 10 is shown in the Figures as being divided into two sub areas 12a, 12b but it is to be understood that the dosage unit 10 may include any desired number of sub areas 12a, 12b. The burstable membrane 14 is seen in phantom in FIG. 1. It is preferred that the membrane 14 be made of a material that, while flexible, is able to be ruptured by way of manual pressure, as shown in FIG. 3.

With reference to FIG. 2, the various layers 16 comprising the dosage unit 10 are seen. The layers 16 comprising the dosage unit 10 are typical to dosage units such as these and preferably include an occlusive, removable backing layer 18, a permeable porous membrane 20 having two surfaces 20a, 20b, a pharmaceutical dosage layer 22 microdispersed on surface 20b, adhesive means 24 on the surface 20a of the porous membrane 20, and an impermeable coating 26.

The removable backing layer 18 is preferably substantially impervious to the pharmaceutical components 30 and the dosage pharmaceutical 31 to be delivered, and extends coextensively with the dosage unit 10 prior to removal. In a preferred embodiment, and as seen in the Figures, the backing layer 18 is removably adhered to surface 20a of the permeable membrane 20 by way of a biologically acceptable adhesive means 24 on surface 20a. The adhesive 24 also serves to adhere the dosage unit 10 to the skin of the patient receiving the dosage (see FIG. 5) and may be over the entire surface 20a of the porous membrane 20, as seen in FIG. 2, or may define an adhesive free area (not shown). Any of the well-known dermatologically acceptable pressure-sensitive adhesives may be used in accordance with this invention, however the adhesive means 24 must additionally exhibit ability to allow pharmaceutical migration therethrough.

The permeable membranous material to be used in the permeable membrane 20 is known in the art and is best described as a plurality of conjoining porous particles which provide a supporting structure while providing a dispersion of microscopic sized interconnecting pores. In order to obtain optimal results, the membrane 20 used for a particular pharmaceutical should be chosen as having the configuration and pore size necessary to achieve the desired release rate for the dosage pharmaceutical 31 to be administered. In addition, the membrane 20 used must be chemically resistant to the dosage pharmaceutical 31 to be administered and non-toxic to the patient receiving the dosage unit 10.

The pharmaceutical compartment layer 22 is preferably divided into a plurality of sub areas, seen as 12a, 12b in these views, the number of sub areas 12a, 12b being determined by the number of individual pharmaceutical components 30 that must be mixed to form the dosage pharmaceutical 31 at time of use. This feature represents a departure from prior, compartmentalized dosage units, which have been constructed having individually formed wells or chambers. The present invention by comparison simply divides a single, large pharmaceutical compartment layer 22 into areas 12a, 12b by way of a burstable or rupturable membrane 14. As seen particularly in FIGS. 3 and 4, this arrangement allows simple manual pressure to rupture the membrane 14 and allow the pharmaceutical components 30 to be mixed to unite and activate as a dosage pharmaceutical 31. As seen particularly in the view of FIG. 4, the burst, fragmented membrane 14 includes distal end portions 28, whose action further agitates the now intermingled pharmaceutical components 30. The flaying action of the distal end portions 28 helps to ensure adequate mixing, and minimizes spot concentrations of unmixed components 30.

As seen particularly in the views of FIGS. 3 and 4, the pharmaceutical components 30 are preferably pigmented to give a visual indication to the user of adequate mixture upon use. In these views, one pharmaceutical component is depicted in dotted shading while the other is in dashed lines. The mixed dosage pharmaceutical 31 is shown as cross hatch. These Figures serve to illustrate the visual indication two indicator colors give when adequately intermingled. While it is preferred that the pharmaceutical components 30 be provided with indicator pigmentation, it is to understood that it is within the scope of the present invention to include components 30 without indicator pigmentation.

The above-described embodiments of this invention are merely descriptive of its principles and are not to be limited. The scope of this invention instead shall be determined from the scope of the following claims, including their equivalents.

What is claimed is:

1. A dermal or transdermal dosage unit for administering a controlled amount of a dosage pharmaceutical to a skin or mucous membrane comprising:
a removable backing layer that is substantially impervious to said pharmaceutical;
a biologically acceptable adhesive layer for securing said dosage unit to said skin or mucous membrane;
a compartment layer containing a measured amount of said dosage pharmaceutical, said dosage pharmaceutical comprising a mixture of a plurality of pharmaceuticals wherein said plurality of pharmaceuticals must be mixed at time of administration of said dosage unit;
a protective coating layer which is substantially impervious to said pharmaceutical; and
said compartment layer initially comprising adjacent subcompartments separated from one another by at least one continuous, uninterrupted impervious burstable membranous partition extending through said compartment layer until such time as said burstable impervious membranous partition has been ruptured to provide flowable access of the pharmaceutical contained in each subcompartment to be intermingled and dispersed throughout said compartment layer to thereby comprise said dosage pharmaceutical.

2. A dermal or transdermal dosage unit for administering a controlled amount of a dosage pharmaceutical to a skin or mucous membrane comprising:
a removable backing layer that is substantially impervious to said pharmaceutical;
a biologically acceptable adhesive layer for securing said dosage unit to said skin or mucous membrane;
a compartment layer containing a measured amount of said dosage pharmaceutical, said dosage pharmaceutical comprising a mixture of a plurality of pharmaceuticals wherein said plurality of pharmaceuticals must be mixed at time of administration of said dosage unit;
a protective coating layer which is substantially impervious to said pharmaceutical;
said compartment layer initially comprising adjacent subcompartments separated from one another by at least one continuous, uninterrupted impervious burstable membranous partition extending through said compartment layer until such time as said burstable impervious membranous partition has been ruptured to provide flowable access of the pharmaceutical contained in each subcompartment to be intermingled and dispersed throughout said compartment layer to thereby comprise said dosage pharmaceutical; and
wherein said continuous, uninterrupted impervious burstable membranous partition, when ruptured comprises at least two distal end portions, said distal end portions providing flagellating action when manipulated for intermingling of said dosage pharmaceuticals.

3. A dermal or transdermal dosage unit for administering a controlled amount of a dosage pharmaceutical to a skin or mucous membrane comprising:
a removable backing layer that is substantially impervious to said pharmaceutical;
a biologically acceptable adhesive layer for securing said dosage unit to said skin or mucous membrane;
a compartment layer containing a measured amount of said dosage pharmaceutical, said dosage pharmaceutical comprising a mixture of a plurality of pharmaceuticals wherein said plurality of pharmaceuticals must be mixed at time of administration of said dosage unit;
a protective coating layer which is substantially impervious to said pharmaceutical;
said compartment layer initially comprising adjacent subcompartments separated from one another by at least one continuous, uninterrupted impervious burstable membranous partition extending through said compartment layer until such time as said burstable impervious membranous partition has been ruptured to provide flowable access of the pharmaceutical contained in each subcompartment to be intermingled and dispersed throughout said compartment layer to thereby comprise said dosage pharmaceutical; and
wherein each of said pharmaceuticals contained in each subcompartment is of an initial color distinct from one another and capable of combining to form a color combination which is distinct from each of said initial colors.

4. A dermal or transdermal dosage unit for administering a controlled amount of a dosage pharmaceutical to a skin or mucous membrane comprising:
a removable backing layer that is substantially impervious to said pharmaceutical;
a biologically acceptable adhesive layer for securing said dosage unit to said skin or mucous membrane;
a compartment layer containing a measured amount of said dosage pharmaceutical, said dosage pharmaceutical comprising a mixture of a plurality of pharmaceuticals wherein said plurality of pharmaceuticals must be mixed at time of administration of said dosage unit;
a protective coating layer which is substantially impervious to said pharmaceutical;
said compartment layer initially comprising adjacent subcompartments separated from one another by at least one continuous, uninterrupted impervious burstable membranous partition extending through said compartment layer until such time as said burstable impervious membranous partition has been ruptured to provide flowable access of the pharmaceutical contained in each subcompartment to be intermingled and dispersed throughout said compartment layer to thereby comprise said dosage pharmaceutical, wherein said continuous, uninterrupted impervious burstable membranous partition, when ruptured, comprises at least two distal end portions, said distal end portions providing flagellating action when manipulated for intermingling of said dosage pharmaceuticals; and
wherein each of said pharmaceuticals contained in each subcompartment is of an initial color distinct from one another and capable of combining to form a color combination which is distinct from each of said initial colors.
